(19) United States
(12) Patent Application Publication
(10) Pub. No.: US 2025/0057421 A1
(43) Pub. Date: Feb. 20, 2025

(54) SYSTEMS AND METHODS FOR GENERATING NEURO-OPHTHALMIC EXAMINATIONS

(71) Applicant: Thomas Jefferson University, Philadelphia, PA (US)

(72) Inventors: James J. Evans, Newtown Square, PA (US); Nikolaos Mouchtouris, Philadelphia, PA (US); Vadim Geyfman, Mariton, NJ (US); Stephane Krumenacker, New York, NY (US)

(21) Appl. No.: 18/723,210

(22) PCT Filed: Dec. 22, 2022
(86) PCT No.: PCT/US2022/053877

§ 371 Date: Jun. 21, 2024

Related U.S. Application Data
(60) Provisional application No. 63/293,454, filed on Dec. 23, 2021.

(51) Int. Cl. A61B 3/18; A61B 5/00; G16H 10/20; G16H 10/60; G16H 15/00; G16H 40/63

(52) U.S. Cl. CPC A61B 3/18; G16H 10/20; G16H 10/60; G16H 15/00; G16H 40/63; A61B 5/40

(57) ABSTRACT

A computer-implemented method is provided herein. One aspect of the invention provides a computer-implemented method including: (a) receiving a set of neuro-ophthalmic examination results for a patient; (b) identifying, from the set of neuro-ophthalmic examination results, a set of patient characteristics; and (c) adjusting one or more parameters of subsequent electronic neuro-ophthalmic examinations based on the set of patient characteristics.

### CROSS-REFERENCE TO RELATED APPLICATION

The present application claims priority to U.S. Provisional Patent Application No. 63/293,454, filed Dec. 23, 2021, which is incorporated herein by reference in its entirety.

### BACKGROUND

Conventional medical practices are often limited to in-person meetings between a patient and a medical professional. This can be a great burden on a patient, particularly where the patient lives a significant distance away from a corresponding medical center, or if the patient's medical condition requires numerous patient-medical professional interactions.

Telemedicine offers the ability to reduce these patient burdens. However, while advances have been made in telemedicine, conventional telemedicine platforms are limited in their ability to perform certain examinations. For example, it is difficult to provide remote generic neuro-ophthalmic examinations, as the patient may experience one or more neuro-ophthalmic examinations condition or symptoms unique to the patient.

### SUMMARY

One aspect of the invention provides a computer-implemented method including: (a) receiving a set of neuro-ophthalmic examination results for a patient; (b) identifying, from the set of neuro-ophthalmic examination results, a set of patient characteristics; and (c) adjusting one or more parameters of subsequent electronic neuro-ophthalmic examinations based on the set of patient characteristics.

Another aspect of the invention provides a system for generating a set of neuro-ophthalmic examinations. The system includes a display screen. The system also includes one or more processors configured to execute a set of instructions that cause the one or more processors to: (a) receive a set of neuro-ophthalmic examination results for a patient; (b) identify, from the set of neuro-ophthalmic examination results, a set of patient characteristics; and (c) adjust one or more parameters of the set of electronic neuro-ophthalmic examinations based on the set of patient characteristics.

Another aspect of the invention provides a computer-readable medium for generating a neuro-ophthalmic examination report. The computer-readable medium includes one or more processors. The computer-readable medium also includes memory. The computer-readable medium also includes a set of instructions stored in the memory that, when executed by the one or more processors, cause the one or more processors to: (a) receive a set of neuro-ophthalmic examination results for a patient; (b) identify, from the set of neuro-ophthalmic examination results, a set of patient characteristics; and (c) adjust one or more parameters of the set of electronic neuro-ophthalmic examinations based on the set of patient characteristics.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a system for generating a neuro-ophthalmic examination according to an embodiment of the present disclosure.

FIG. 2 depicts a server for generating a neuro-ophthalmic examination according to an embodiment of the present disclosure.

FIGS. 3-9 depicts a workflow for generating a neuro-ophthalmic examination according to embodiment of the present disclosure.

FIG. 10 depicts a process flow for generating a neuro-ophthalmic examination according to an embodiment of the present disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Systems, devices, and associated methods to implement neuro-ophthalmic examinations, track progress of underlying neuro-ophthalmic conditions of a patient, and adjusting subsequent neuro-ophthalmic examinations for the patient are described herein. The systems, devices, and methods described herein can include a series of questions and assessments that test the user's cranial nerve and neuro-ophthalmic functions. Each assessment can tests a different aspect of a user's condition, but the findings of each test can assist in refining and improving the subsequent tests. The series of tests can include the following: questionnaires for patient symptoms and past medical history; facial sensation exams; visual acuity exams; visual fields exams; color blindness exams; Amsler grid exams; cranial nerve video recording exams; hearing tests; arm/leg strength tests; gait tests; limb sensation tests; and double vision tests.

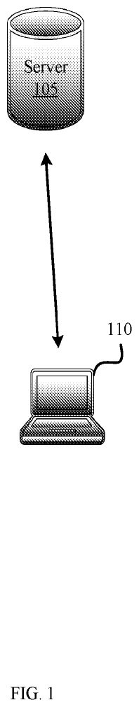

FIG. 1 depicts a system for generating neuro-ophthalmic examinations according to an embodiment of the present disclosure. The system can include a server 105 and a computing device 110.

The server 105 can store instructions for performing a neuro-ophthalmic examination. In some cases, the server 105 can also include a set of processors that execute the set of instructions. Further, the server 105 can be any type of server capable of storing and/or executing instructions, for example, an application server, a web server, a proxy server, a file transfer protocol (FTP) server, and the like. In some cases, the server 105 can be a part of a cloud computing architecture, such as a Software as a Service (Saas), Development as a Service (DaaS), Data as a Service (DaaS), Platform as a Service (PaaS), and Infrastructure as a Service (IaaS).

A computing device 110 can be in electronic communication with the server 105 and can display the neuro-ophthalmic examination to a user. The computing device 110 can include a display for displaying the neuro-ophthalmic examination, and a user input device, such as a mouse, keyboard, or touchpad, for logging and transmitting user input corresponding to the neuro-ophthalmic examination. In some cases, the computing device 110 can include a set of processors for executing the neuro-ophthalmic examination (e.g., from instructions stored in memory). Examples of a computing device include, but are not limited to, a personal computer, a laptop, a tablet, a cellphone, a personal digital assistant, an e-reader, a mobile gaming device, and the like.

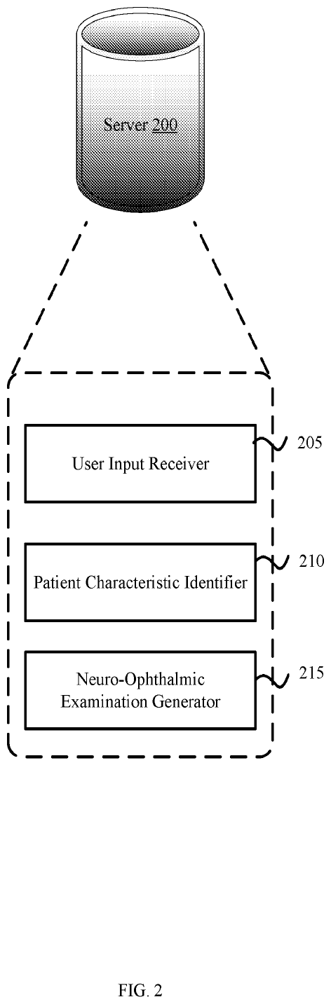

FIG. 2 depicts a server 200 for generating neuro-ophthalmic examinations according to an embodiment of the present disclosure. The server can be an example of the server 105 as discussed with reference to FIG. 1. The server 200 can include a user input receiver 205, a patient characteristic identifier 210, and, an object position determination component 215.

The user input receiver 205 can receive user input from the computing device. For example, the user input can be a mouse click, a keyboard click, a touch on a touchpad, and the like. The user input receiver 210 can receive the user input and log different parameters of the user input. For example, the user input receiver 210 can identify a timestamp of the user input, the type of user input (e.g., mouse click, keyboard click, etc.) and the like. The server 200 may store the user input in memory.

In some cases, the user input can correspond to various neuro-ophthalmic examinations implemented by the system (e.g., the system 100 of FIG. 1). For example, a user may be asked to provide user input corresponding to one or more neuro-ophthalmic examinations, such as questionnaires for patient symptoms and past medical history, facial sensation exams, visual acuity exams, visual fields exams, color blindness exams, Amsler grid exams, cranial nerve video recording exams, hearing tests, arm/leg strength tests, gait tests, limb sensation tests, double vision tests, and the like. The user may respond or interact with the neuro-ophthalmic examinations via the user input receiver 205.

The patient characteristic identifier 210 can identify one or more characteristics of the user based on the received user input. In some cases the patient characteristic identifier 210 can determine one or more conditions or symptoms the user experiences based on the received user input. In some cases, conditions or symptoms may correspond to neuro-ophthalmic conditions or diseases. For example, some conditions the patient characteristic identifier 210 can identify for a user can include macular degeneration, glaucoma, macular edema, chorioretinopathy, optic neuritis, ocular hypertension, optic neuropathy, and the like.

The neuro-ophthalmic examination generator 215 adjust one or more parameters of an electronic neuro-ophthalmic examination based on the identified patient characteristics. For example, if a patient is determined to experience double vision in a particular angle of vision, the neuro-ophthalmic examination generator 215 may adjust measurements taken of another neuro-ophthalmic examination for the patient in the proximity of the angle of vision, such as discounting user input received during the subsequent neuro-ophthalmic examination at that given angle. In other cases, the neuro-ophthalmic examination generator 215 may increase a weight of received user input for a given neuro-ophthalmic examination compared to other examinations, for example, if a particular neuro-ophthalmic examination has a larger correlation to a neuro-ophthalmic condition the patient has compared to another neuro-ophthalmic examination that detects dissimilar conditions.

In some other cases, characteristics of how a neuro-ophthalmic examination is displayed can be adjusted based on identified patient characteristics. For example, if a patient is determined to be near-sighted from a previous examination, future examinations may be implemented with a larger font size, or a magnified image on the computing device display. In another example, if a patient is determined to be color blind, color parameters for subsequent examinations may be adjusted.

The parameter adjustment may be based on a patient characteristic exceeding a threshold. For example, in the example where the user is determined to be near-sighted, the font or magnification of subsequent examinations may be initiated if the user's eye prescription values exceed a predefined threshold (e.g., −2.25, and the like).

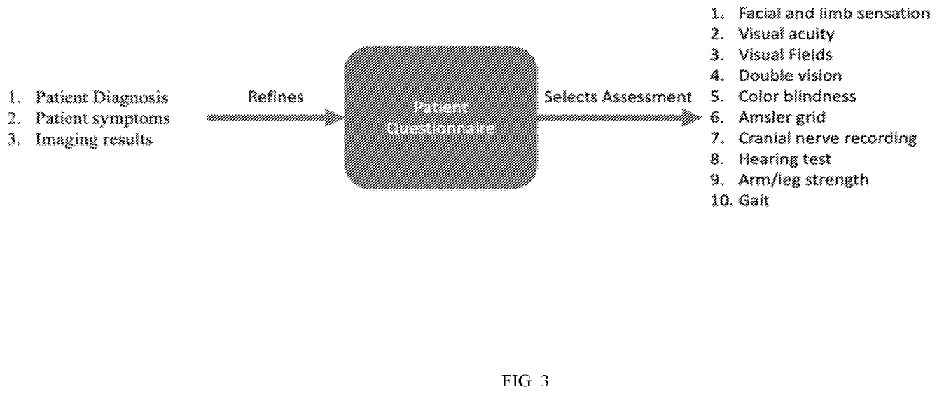

FIG. 3 depicts a workflow process for generating a neuro-ophthalmic examination according to an embodiment of the present disclosure. In this embodiment, the neuro-ophthalmic examination results can include a patient neuro-ophthalmic diagnosis, a patient symptom, an imaging result, or a combination thereof. The system can identify patient demographics from the results. The system can further adjust subsequent examination parameters of: facial and limb sensation parameter, a visual acuity parameter, a visual field parameter, a double vision parameter, a color blindness parameter, an Amsler grid parameter, a cranial nerve recording parameter, a hearing test parameter, an arm or leg strength parameter, a gait parameter, and the like.

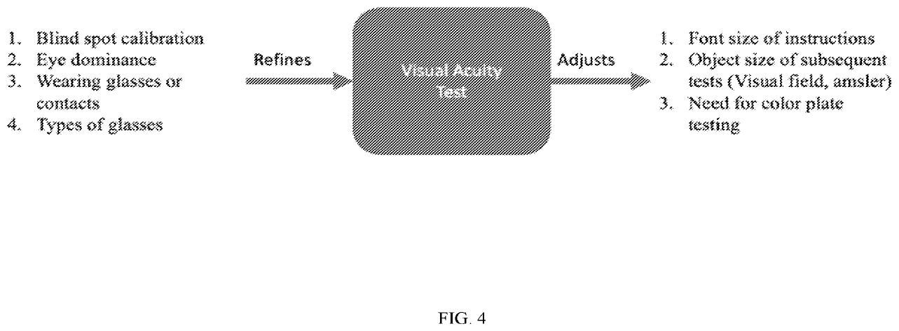

FIG. 4 depicts a workflow process for generating a neuro-ophthalmic examination according to an embodiment of the present disclosure. In this embodiment, the neuro-ophthalmic examination results can include a blind spot calibration result, an eye dominance result, a glasses or contact lens wearing result, a type of eyeglasses, and the like. The system can identify a visual acuity parameter for the user from the examination results. The system can further adjust subsequent examination parameters of: an instruction font size parameter, an object size parameter, a color plate parameter, and the like.

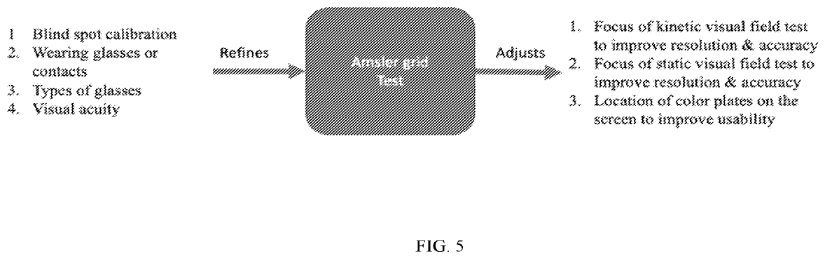

FIG. 5 depicts a workflow process for generating a neuro-ophthalmic examination according to an embodiment of the present disclosure. In this embodiment, the neuro-ophthalmic examination results can include a blind spot calibration result, a glasses or contact lens wearing result, a type of eyeglasses, a visual acuity result, and the like. The system can identify an Amsler grid parameter from the examination results. The system can further adjust subsequent parameters of: a focus of kinetic field test parameter, a focus of static visual field test parameter, a color plate location parameter, and the like.

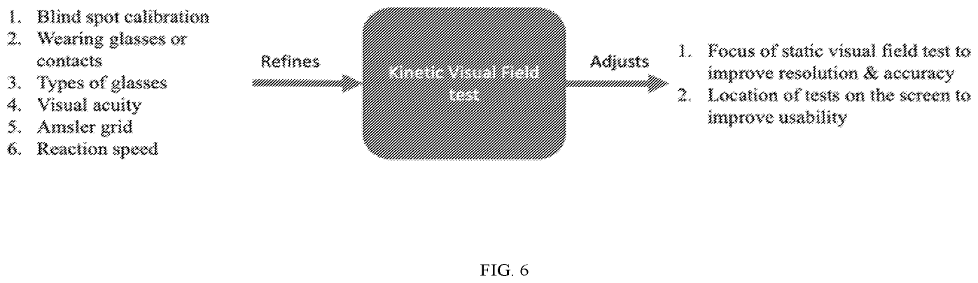

FIG. 6 depicts a workflow process for generating a neuro-ophthalmic examination according to an embodiment of the present disclosure. In this embodiment, the neuro-ophthalmic examination results can include a blind spot calibration result, a glasses or contact lens wearing result, a type of eyeglasses, a visual acuity result, an Amsler grid test result, a reaction speed test result. The system can identify a kinetic visual field test parameter from the examination results. The system can further adjust subsequent parameters of: a static field test focus parameter, a test display location parameter, and the like.

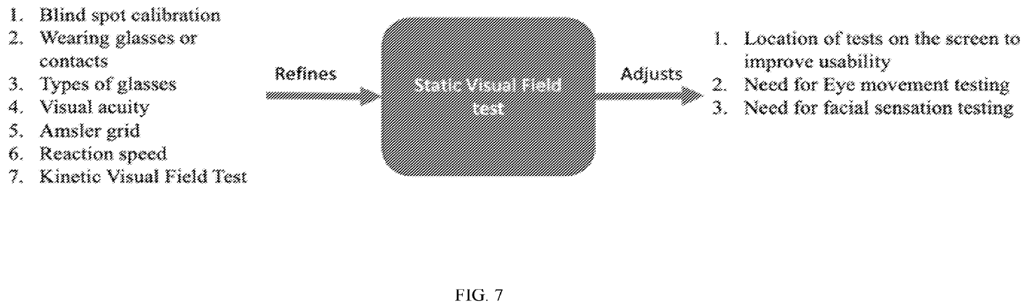

FIG. 7 depicts a workflow process for generating a neuro-ophthalmic examination according to an embodiment of the present disclosure. In this embodiment, the neuro-ophthalmic examination results can include a blind spot calibration result, a glasses or contact lens wearing result, a type of eyeglasses, a visual acuity result, an Amsler grid test result, a reaction speed test result, a kinetic visual field test result, and the like. The system can identify a static visual field test parameter from the examination results. The system can further adjust subsequent parameters of: a test display location parameter, an eye movement test requirement parameter, a facial sensation test requirement parameter, and the like.

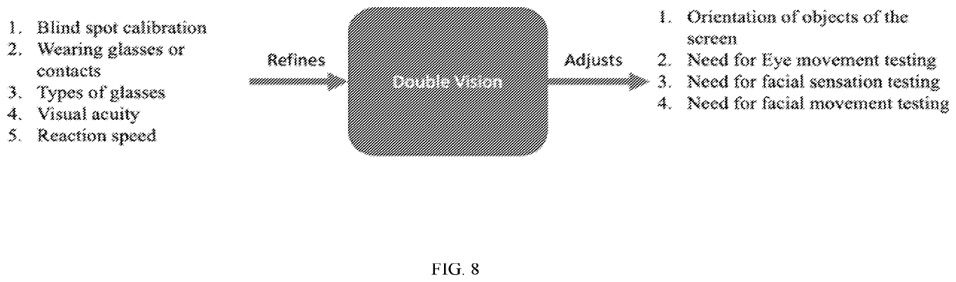

FIG. 8 depicts a workflow process for generating a neuro-ophthalmic examination according to an embodiment of the present disclosure. In this embodiment, the neuro-ophthalmic examination results can include a blind spot calibration result, a glasses or contact lens wearing result, a type of eyeglasses, a visual acuity result, a reaction speed test result, and the like. The system can identify a double vision parameter based on the examination results. The system can further adjust subsequent parameters of: an object orientation parameter, an eye movement test requirement parameter, a facial sensation test requirement parameter, a facial movement test requirement parameter, and the like.

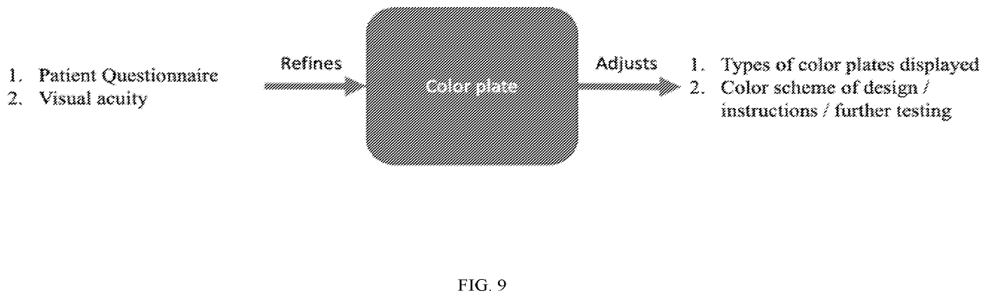

FIG. 9 depicts a workflow process for generating a neuro-ophthalmic examination according to an embodiment of the present disclosure. In this embodiment, the neuro-ophthalmic examination results can include patient questionnaire results, visual acuity results, and the like. The system can identify a color plate parameter for the user from the examination results. The system can further adjust subsequent parameters of: a color plate color type parameter, a color scheme instruction parameter, and the like.

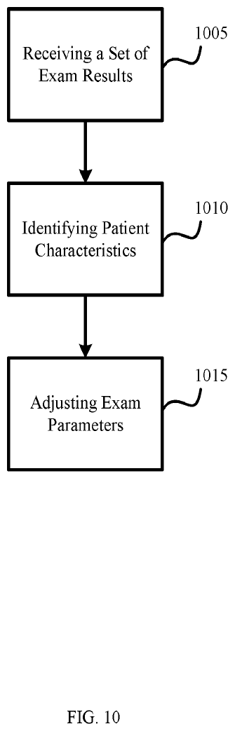

FIG. 10 depicts a process flow for generating a neuro-ophthalmic examination according to an embodiment of the present disclosure. The process flow can be implemented by system 100 of FIG. 1. In some cases, the process flow can be implemented by computing device 110 of FIG. 1.

At Step 1005, a set of neuro-ophthalmic examination results for a patient can be received. In some cases, the results can be received via computer mouse, a touchscreen, a microphone, a keyboard, a video camera, or a combination thereof. The results can be received via a user input receiver 210 of FIG. 2.

At Step 1010, a set of patient characteristics can be identified from the set of neuro-ophthalmic examination results, such as those described in FIGS. 3-9. The identification can be made by the patient characteristic identifier 210 of FIG. 2.

At Step 1015, one or more parameters of subsequent electronic neuro-ophthalmic examinations can be adjusted based on the set of patient characteristics, such as those described in FIGS. 3-9. The adjustment can be made by the neuro-ophthalmic examination generator 215 of FIG. 2.

### Study

In a study of the present disclosure, 68 participants with an average age of 52.1±16.2 years old, underwent an algorithm-driven responsive cranial nerve and neuro-ophthalmic assessment, in accordance with certain exemplary embodiments of the present disclosure. Based on the user's diagnosis and the user's answers on the triaging questionnaire, the user was asked to take one or more of the cranial nerve and/or neuro-ophthalmic assessments included in exemplary software (facial and limb sensation, visual acuity, visual fields, double vision, color blindness test, Amsler grid, smile symmetry, eyelid closure, shoulder elevation, head rotation, tongue protrusion, eye muscle range of motion, hearing test) in accordance with certain embodiments of the present disclosure.

### Exemplary Elements of Responsive Design

An example of software implementing certain embodiments of the present disclosure is described herein. This description is exemplary in nature and not intended to limit the disclosure based on specific numerical values described herein.

Users who answer in the questionnaire that have visual complaints are asked which eye is affected the most. Based on that information, all vision testing can be initiated with the least affected eye so the user becomes familiar with the assessments. Then the user can proceed to test their more affected eye. Such a step can significantly improve study compliance and accuracy of results.

Based on the user's performance on a visual acuity test, the size of the dot displayed in the static and kinetic visual field testing is adjusted accordingly. If the user's vision is better than 20/70, the dot size is 4 mm². If the visual acuity is 20/100-20/200, the dot size can be increased to 32 mm². If the visual acuity is worse than 20/200, the dot size can be increased to 64 mm². Similar responsive sizing changes can be made throughout the software. If the visual acuity is worse than 20/200, color plate testing is not offered to the user due to the user's poor vision.

Based on a blind spot calibration functionality, the dimensions of each assessment (e.g., visual acuity, static visual field, kinetic visual field, double vision, Amsler grid, etc.) are adjusted to maintain accuracy and precision between each time a user undergoes testing.

Based on the user's performance on a color plate test, the color palette used throughout the software (e.g., instructions, future testing) is adjusted to ensure usability and testing compliance.

If the user responds ‘yes’ to questions regarding facial pain, facial numbness, muscle twitching, difficulty swallowing, hoarseness, or throat/mouth pain, the user can be asked to undergo a facial sensation assessment, smile symmetry, eyelid closure, shoulder elevation, head rotation, and the like.

If the user responds ‘yes’ to questions regarding difficulty hearing, the user can be asked to undergo a hearing test.

If the user responds ‘yes’ to questions regarding blurry or double vision, the user can be asked to undergo an eye muscle range of motion assessment and a double vision assessment in addition to visual acuity and visual field testing.

### ENUMERATED EMBODIMENTS

The following enumerated embodiments are provided, the numbering of which is not to be construed as designating levels of importance.

Embodiment 1 provides a computer-implemented method including:
receiving a set of neuro-ophthalmic examination results for a patient;
identifying, from the set of neuro-ophthalmic examination results, a set of patient characteristics; and
adjusting one or more parameters of subsequent electronic neuro-ophthalmic examinations based on the set of patient characteristics.

Embodiment 2 provides the computer-implemented method of embodiment 1, wherein the set of neuro-ophthalmic examination results includes a patient neuro-ophthalmic diagnosis, a patient symptom, an imaging result, or a combination thereof, and wherein the one or parameters include a facial and limb sensation parameter, a visual acuity parameter, a visual field parameter, a double vision parameter, a color blindness parameter, an Amsler grid parameter, a cranial nerve recording parameter, a hearing test parameter, an arm or leg strength parameter, a gait parameter, or a combination thereof.

Embodiment 3 provides the computer-implemented method of any one of embodiments 1-2, wherein the set of neuro-ophthalmic examination results includes a blind spot calibration result, an eye dominance result, a glasses or contact lens wearing result, a type of eyeglasses, or a combination thereof, and wherein the one or more parameters include an instruction font size parameter, an object size parameter, a color plate parameter, or a combination thereof.

Embodiment 4 provides the computer-implemented method of any one of embodiments 1-3, wherein the set of neuro-ophthalmic examination results includes a blind spot calibration result, a glasses or contact lens wearing result, a type of eyeglasses, a visual acuity result, or a combination thereof, and wherein the one or more parameters include a focus of kinetic field test parameter, a focus of static visual field test parameter, a color plate location parameter, or a combination thereof.

Embodiment 5 provides the computer-implemented method of any one of embodiments 1-4, wherein the set of neuro-ophthalmic examination results includes a blind spot calibration result, a glasses or contact lens wearing result, a type of eyeglasses, a visual acuity result, an Amsler grid test result, a reaction speed test result, or a combination thereof, and wherein the one or more parameters include a static field test focus parameter, a test display location parameter, or a combination thereof.

Embodiment 6 provides the computer-implemented method of any one of embodiments 1-5, wherein the set of neuro-ophthalmic examination results includes a blind spot calibration result, a glasses or contact lens wearing result, a type of eyeglasses, a visual acuity result, an Amsler grid test result, a reaction speed test result, a kinetic visual field test result, or a combination thereof, and wherein the one or more parameters include a test display location parameter, an eye movement test requirement parameter, a facial sensation test requirement parameter, or a combination thereof.

Embodiment 7 provides the computer-implemented method of any one of embodiments 1-6, wherein the set of neuro-ophthalmic examination results includes a blind spot calibration result, a glasses or contact lens wearing result, a type of eyeglasses, a visual acuity result, a reaction speed test result, or a combination thereof, and wherein the one or more parameters include an object orientation parameter, an eye movement test requirement parameter, a facial sensation test requirement parameter, a facial movement test requirement parameter, or a combination thereof.

Embodiment 8 provides the computer-implemented method of any one of embodiments 1-7, wherein the set of neuro-ophthalmic examination results comprises patient questionnaire results, visual acuity results, or a combination thereof, and wherein the one or more parameters comprise a color plate color type parameter, a color scheme instruction parameter, or a combination thereof.

Embodiment 9 provides the computer-implemented method of any one of embodiments 1-8, wherein the subsequent electronic neuro-ophthalmic examinations include a neuro-ophthalmic patient questionnaire, a visual acuity test, an Amsler grid test, a kinetic visual field test, a static visual field test, a double vision test, a color plate test, or a combination thereof.

Embodiment 10 provides a system for generating a set of neuro-ophthalmic examinations, including:
a display screen; and
one or more processors configured to execute a set of instructions that cause the one or more processors to:
   receive a set of neuro-ophthalmic examination results for a patient;
   identify, from the set of neuro-ophthalmic examination results, a set of patient characteristics; and
   adjust one or more parameters of the set of electronic neuro-ophthalmic examinations based on the set of patient characteristics.

Embodiment 11 provides a computer-readable medium for generating a neuro-ophthalmic examination report, including:
one or more processors;
memory; and
a set of instructions stored in the memory that, when executed by the one or more processors, cause the one or more processors to:
   receive a set of neuro-ophthalmic examination results for a patient;
   identify, from the set of neuro-ophthalmic examination results, a set of patient characteristics; and
   adjust one or more parameters of the set of electronic neuro-ophthalmic examinations based on the set of patient characteristics.

Embodiment 12 provides the system of embodiment 10, wherein the system is configured and adapted to implement any of the methods of embodiments 1-9.

Embodiment 13 provides the computer-readable medium of embodiment 11, wherein the computer-readable medium is configured and adapted to implement any of the methods of embodiments 1-9.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method comprising:
receiving a set of neuro-ophthalmic examination results for a patient;
identifying, from the set of neuro-ophthalmic examination results, a set of patient characteristics; and
adjusting one or more parameters of subsequent electronic neuro-ophthalmic examinations based on the set of patient characteristics.

2. The computer-implement method of claim 1, wherein the set of neuro-ophthalmic examination results comprises a patient neuro-ophthalmic diagnosis, a patient symptom, an imaging result, or a combination thereof, and wherein the one or parameters comprise a facial and limb sensation parameter, a visual acuity parameter, a visual field parameter, a double vision parameter, a color blindness parameter, an Amsler grid parameter, a cranial nerve recording parameter, a hearing test parameter, an arm or leg strength parameter, a gait parameter, or a combination thereof.

3. The computer-implemented method of claim 1, wherein the set of neuro-ophthalmic examination results comprises a blind spot calibration result, an eye dominance result, a glasses or contact lens wearing result, a type of eyeglasses, or a combination thereof, and wherein the one or more parameters comprise an instruction font size parameter, an object size parameter, a color plate parameter, or a combination thereof.

4. The computer-implemented method of claim 1, wherein the set of neuro-ophthalmic examination results comprises a blind spot calibration result, a glasses or contact lens wearing result, a type of eyeglasses, a visual acuity result, or a combination thereof, and wherein the one or more parameters comprise a focus of kinetic field test parameter, a focus of static visual field test parameter, a color plate location parameter, or a combination thereof.

5. The computer-implemented method of claim 1, wherein the set of neuro-ophthalmic examination results comprises a blind spot calibration result, a glasses or contact lens wearing result, a type of eyeglasses, a visual acuity result, an Amsler grid test result, a reaction speed test result, or a combination thereof, and wherein the one or more parameters comprise a static field test focus parameter, a test display location parameter, or a combination thereof.

6. The computer-implemented method of claim 1, wherein the set of neuro-ophthalmic examination results comprises a blind spot calibration result, a glasses or contact lens wearing result, a type of eyeglasses, a visual acuity result, an Amsler grid test result, a reaction speed test result, a kinetic visual field test result, or a combination thereof, and wherein the one or more parameters comprise a test display location parameter, an eye movement test requirement parameter, a facial sensation test requirement parameter, or a combination thereof.

7. The computer-implemented method of claim 1, wherein the set of neuro-ophthalmic examination results comprises a blind spot calibration result, a glasses or contact lens wearing result, a type of eyeglasses, a visual acuity result, a reaction speed test result, or a combination thereof, and wherein the one or more parameters comprise an object orientation parameter, an eye movement test requirement parameter, a facial sensation test requirement parameter, a facial movement test requirement parameter, or a combination thereof.

8. The computer-implemented method of claim 1, wherein the set of neuro-ophthalmic examination results comprises patient questionnaire results, visual acuity results, or a combination thereof, and wherein the one or more parameters comprise a color plate color type parameter, a color scheme instruction parameter, or a combination thereof.

9. The computer-implemented method of claim 1, wherein the subsequent electronic neuro-ophthalmic examinations comprise a neuro-ophthalmic patient questionnaire, a visual acuity test, an Amsler grid test, a kinetic visual field test, a static visual field test, a double vision test, a color plate test, or a combination thereof.

10. A system for generating a set of neuro-ophthalmic examinations, comprising:
a display screen; and
one or more processors configured to execute a set of instructions that cause the one or more processors to:
receive a set of neuro-ophthalmic examination results for a patient;
identify, from the set of neuro-ophthalmic examination results, a set of patient characteristics; and
adjust one or more parameters of the set of electronic neuro-ophthalmic examinations based on the set of patient characteristics.

11. A computer-readable medium for generating a neuro-ophthalmic examination report, comprising:
one or more processors;
memory; and
a set of instructions stored in the memory that, when executed by the one or more processors, cause the one or more processors to:
receive a set of neuro-ophthalmic examination results for a patient;
identify, from the set of neuro-ophthalmic examination results, a set of patient characteristics; and
adjust one or more parameters of the set of electronic neuro-ophthalmic examinations based on the set of patient characteristics.
